(19) United States
(12) Patent Application Publication
(10) Pub. No.: US 2019/0134382 A1
(43) Pub. Date: May 9, 2019

(54) METHOD AND SYSTEM FOR ADJUSTING STIMULATION THERAPY

(71) Applicant: PACESETTER, INC., Sylmar, CA (US)

(72) Inventors: Filippo Agnesi, Plano, TX (US); Prasanna Kumar, Frisco, TX (US)

(21) Appl. No.: 15/806,676

(22) Filed: Nov. 8, 2017

(51) Int. Cl. A61N 1/05; A61N 1/36

(52) U.S. Cl. CPC A61N 1/0551; A61N 1/36062; A61B 5/04001; A61N 1/36071; A61N 1/36135; A61N 1/36132

(57) ABSTRACT

The systems and methods described herein generally relate to spinal cord stimulation (SCS) therapy and more particularly to monitoring the SCS therapy and determining an adjustment to a position of a lead or electrodes delivering the SCS therapy to the patient. The systems and methods deliver a SCS therapy to a portion of an array of electrodes of a lead, and analyze physiological signals measured by a set of sensors relative to each other. The set of sensors are positioned bilaterally on a patient. Further, the systems and methods determine an adjustment in a position of the lead or the SCS therapy based on a relation between the physiological signals.

### BACKGROUND OF THE INVENTION

Embodiments herein generally relate to spinal cord stimulation (SCS) therapy and more particularly to monitoring the SCS therapy and determining an adjustment to a position of a lead or electrodes delivering the SCS therapy to the patient.

Neurostimulation systems are devices that generate electrical pulses and deliver the pulses to nerve tissue to treat a variety of disorders. SCS is the most common type of neurostimulation. In SCS, electrical pulses are delivered to nerve tissue in the spine typically for the purpose of chronic pain control representing a SCS therapy. While a precise understanding of the interaction between the applied electrical energy and the nervous tissue is not fully appreciated, it is known that application of an electrical field to spinal nervous tissue can mask certain types of pain transmitted from regions of the body associated with the stimulated nerve tissue. Applying electrical energy to the spinal cord associated with regions of the body afflicted with chronic pain can induce “paresthesia” (a subjective sensation of numbness or tingling) in the afflicted bodily regions. Paresthesia can effectively mask the transmission of non-acute pain sensations to the brain.

During implantation, a lead of the SCS systems is positioned within a patient. To identify an optimal location for electrode implantation, patient must provide feedback during surgery on which areas of the body are affected by stimulation. Conventional methods require waking the patient from anesthesia and performing a set of stimulations that induce sensory perceptions. The patient identifies the body location affected by the stimulation, which should overlap the painful areas. If the overlap is not achieved, the clinician must move the electrode while the patient is awake. The conventional method is not only cumbersome but is also very stressful for the patient. Moreover the conventional method is time consuming, and the feedback may not be accurate as the patient is potentially confused by the surgical pain and/or still under the influence of sedation to some degree.

A need remains for improved methods and systems for adjusting a position for SCS therapy during implantation of the SCS system.

### SUMMARY

In accordance with an embodiment, a system is provided for electrode monitoring. The system includes a lead having an array of electrodes. The lead is configured to be implanted within an epidural space of a dorsal column of a patient's spine. The system includes a pulse generator (PG) electrically coupled to the lead. The PG is configured to deliver spinal cord stimulation (SCS) therapy. The system includes a set of sensors positioned bilaterally on a patient and configured to acquire physiological signals. The system includes a controller circuit configured to respond to instructions stored on a non-transient computer-readable medium. The controller circuit is configured to deliver the SCS therapy to a portion of the electrodes, analyze physiological signals measured by the set of sensors relative to each other, and determine an adjustment in a position of the lead or the SCS therapy based on a relation between the physiological signals.

In accordance with an embodiment, a method is provided for electrode monitoring. The method includes delivering spinal cord stimulation (SCS) therapy to a portion of an array of electrodes of a lead, and analyzing physiological signals measured by a set of sensors relative to each other. The set of sensors are positioned bilaterally on a patient. The method includes determining an adjustment in a position of the lead or the SCS therapy based on a relation between the physiological signals.

### BRIEF DESCRIPTION OF THE DRAWINGS

FIG. 1 depicts a schematic block diagram of an embodiment of a neurostimulation system.

FIGS. 2A-2I respectively depict stimulation portions of embodiments for inclusion at the distal end of a lead.

FIG. 3 depicts a schematic block diagram of an embodiment of a monitoring system.

FIG. 4 illustrates a graphical representation of an embodiment of one or more sets of anatomically opposed sensors positioned bilaterally on a patient.

FIG. 5 illustrates a flowchart of an embodiment of a method for electrode monitoring.

FIG. 6 illustrates a graphical representation of an embodiment of a portion of symmetric physiological signals.

FIG. 7 illustrates graphical representations of an embodiment of a portion of non-symmetric physiological signals.

FIGS. 8A-D illustrates embodiments of user interface components shown on a graphical user interface on a display.

FIG. 9 illustrates graphical representations of an embodiment of a portion of a plurality of physiological signals.

FIGS. 10A-C illustrates embodiments of user interface components shown on a graphical user interface on a display.

### DETAILED DESCRIPTION

While multiple embodiments are described, still other embodiments of the described subject matter will become apparent to those skilled in the art from the following detailed description and drawings, which show and describe illustrative embodiments of disclosed inventive subject matter. As will be realized, the inventive subject matter is capable of modifications in various aspects, all without departing from the spirit and scope of the described subject matter. Accordingly, the drawings and detailed description are to be regarded as illustrative in nature and not restrictive.

Embodiments herein describe a monitoring system configured to deliver spinal cord stimulation (SCS) therapy to a patient and measure physiological responses intraoperatively to assist the implantation of a stimulation lead within the epidural space. In SCS, the efficacy of the stimulation therapy is related to the implant location of stimulation lead. The position of the electrodes of the lead in a precise manner in caudal or cephalic directions affects the ability to recruit appropriate dorsal fibers of the spinal cord associated with the area of chronic pain of the patient For example, it is known to place electrodes near the T-8 vertebral level to address leg and/or back pain of patient. Also, the position of the stimulation lead or paddle relative to the physiological midline is related to stimulating appropriate dorsal column fibers and avoiding undesired stimulation of other dorsal column or dorsal root fibers. The correct orientation of the electrodes of the lead relative to the physiological midline is also correlated to achieving optimal programming of the SCS parameters. When an array of electrodes is positioned diagonally relative the physiological midline, programming of the SCS parameters is more difficult and, in some cases, proper coverage of SCS cannot be achieved without repositioning the electrodes.

Although it is generally known that these factors have significant effects on the efficacy of the SCS therapy, it is challenging to appropriately place a stimulation lead with electrodes in the proper position. Several known techniques have been applied to attempt to verify the correct position of the stimulation lead. In many implant procedures, the patient is awakened from anesthesia, electrical stimulation is applied to the spinal cord, and the patient provides feedback regarding the patient's perception of the electrical stimulation. If an acceptable response to stimulation is not obtained, the patient is given additional anesthesia to permit repositioning of the electrodes by the surgeon and the trial stimulation process is repeated.

In other cases, neuromonitoring and measurement of physiological signals are performed. However, the use of general neuromonitoring and other measurement equipment relies on a high degree of clinician expertise and experience. Further, the use of known physiological signal monitoring equipment during a stimulation lead implant procedure is cumbersome and complicates the surgical space where the procedure is performed. For these reasons, the monitoring and measurement of physiological signals, although known by some practitioners, is not widely employed for SCS implant procedures.

In some embodiments, sensors are attached to the patient bilaterally on sets of muscle groups including those located in the body region(s) affected by pain. The sensors record one or more physiologic parameters measured during the SCS therapy. The monitoring system is attached to the sensors through one or more interfaces or suitable connections. The monitoring system may conduct wireless communication to obtain data from the sensors. The monitoring system is configured to analyze and/or evaluate the one or more physiological signals of the stimulation evoked response measured by the sensors. The monitoring system is configured to compare the one or more physiological signals to determine a degree of symmetry among sets of sensors located on common group muscles positioned at opposing sides of the body. For example, the monitoring system is configured to compare an amplitude and/or phase of the one or more physiological signals to determine a position and/or a degree symmetry of the lead within the patient. Based on the degree of symmetry, the monitoring system is configured to determine an adjustment to a position of a lead or the electrodes delivering the SCS therapy to the patient. The monitoring system may be configured to generate visualizations configured to indicate to a clinician (e.g., doctor, nurse, and/or the like) information relative to the position of the lead, electrode positioning relative to the patient, actions to be taken by the clinician, and/or the like.

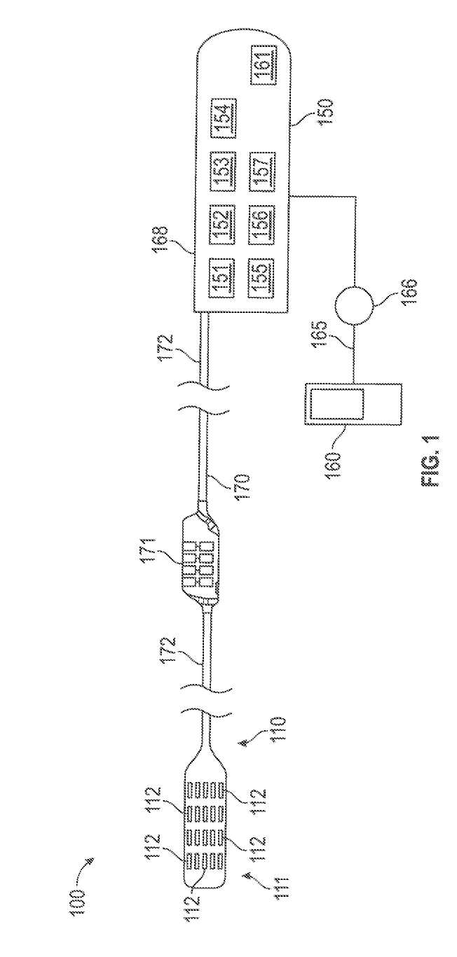

FIG. 1 depicts a schematic block diagram of an embodiment of a neurostimulation (NS) system 100. The NS system 100 is configured to generate electrical pulses (e.g., excitation pulses) for application to tissue of a patient according to some embodiments. For example, the NS system 100 may be adapted to stimulate spinal cord tissue, dorsal root, dorsal root ganglion, peripheral nerve tissue, deep brain tissue, cortical tissue, cardiac tissue, digestive tissue, pelvic floor tissue, and/or any other suitable nerve tissue of interest within a patient's body.

The NS system 100 includes pulse generator (PG) 150 that is adapted to generate electrical pulses for application to tissue of a patient. Pulse generator 150 may be an external pulse generator that is intended to provide trial stimulation to the patient (intraoperatively or during a patient trial outside of medical facilities). Alternatively, pulse generator 150 may be an implantable pulse generator for implant within the patient to provide the long term electrical stimulation therapy. The PG 150 typically comprises a metallic housing or can 158 that encloses a controller circuit 151, pulse generating circuitry 152, a charging coil 153, a battery 154, a communication circuit 155, battery charging circuitry 156, switching circuitry 167, memory 161, and/or the like. The communication circuit 155 may represent hardware that is used to transmit and/or receive data along a bi-directional communication link (e.g., with a clinician programmer 160).

The controller circuit 151 is configured to control the operation of the NS system 100. The controller circuit 151 may include one or more processors, a central processing unit (CPU), one or more microprocessors, or any other electronic component capable of processing input data according to program instructions. Optionally, the controller circuit 151 may include and/or represent one or more hardware circuits or circuitry that include, are connected with, or that both include and are connected with one or more processors, controllers, and/or other hardware logic-based devices. Additionally or alternatively, the controller circuit 151 may execute instructions stored on a tangible and non-transitory computer readable medium (e.g., the memory 161).

The PG 150 may include a separate or an attached extension component 170. The extension component 170 may be a separate component. For example, the extension component 170 may connect with a “header” portion of the PG 150, as is known in the art. If the extension component 170 is integrated with the PG 150, internal electrical connections may be made through respective conductive components. Within the PG 150, electrical pulses are generated by the pulse generating circuitry 152 and are provided to the switching circuitry 157. The switching circuitry 157 connects to outputs of the PG 150. Electrical connectors (e.g., “Bal-Seal” connectors) within the connector portion 171 of the extension component 170 or within the IPG header may be employed to conduct various stimulation pulses. The terminals of one or more leads 110 are inserted within the connector portion 171 or within the IP G header for electrical connection with respective connectors. The pulses originating from the PG 150 are provided to the one or more leads 110. The pulses are then conducted through the conductors of the lead 110 and applied to tissue of a patient via an electrode array 111. Any suitable known or later developed design may be employed for connector portion 171.

The electrode array 111 may be positioned on a paddle structure of the lead 110. For example, the PENTA™ paddle lead (available from Abbott, Plano Tex.) may be employed according to some embodiments. Also, for example, in a planar formation on a paddle structure as disclosed in U.S. Provisional Application No. 61/791,288, entitled, “PADDLE LEADS FOR NEUROSTIMULATION AND METHOD OF DELIVERYING THE SAME” which is expressly incorporated herein by reference. The electrode array 111 includes a plurality of electrodes 112 aligned along corresponding rows and columns. Each of the electrodes 112 are separated by non-conducting portions of the paddle structure, which electrically isolate each electrode 112 from an adjacent electrode 112. The non-conducting portions may include one or more insulative materials and/or biocompatible materials to allow the lead 110 to be implantable within the patient. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane. The electrodes 112 may be configured to emit pulses in an outward direction.

Optionally, the PG 150 may have one or more leads 110 connected via the connector portion 171 of the extension component 170 or within the IPG header. For example, a DRG stimulator, a steerable percutaneous lead, and/or the like. Additionally or alternatively, the electrodes 112 of each lead 110 may be configured separately to emit excitation pulses.

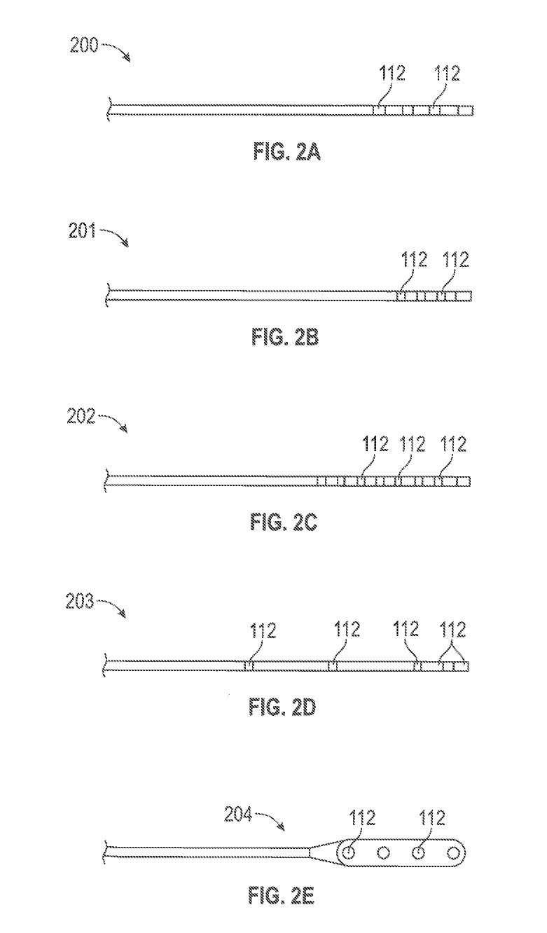

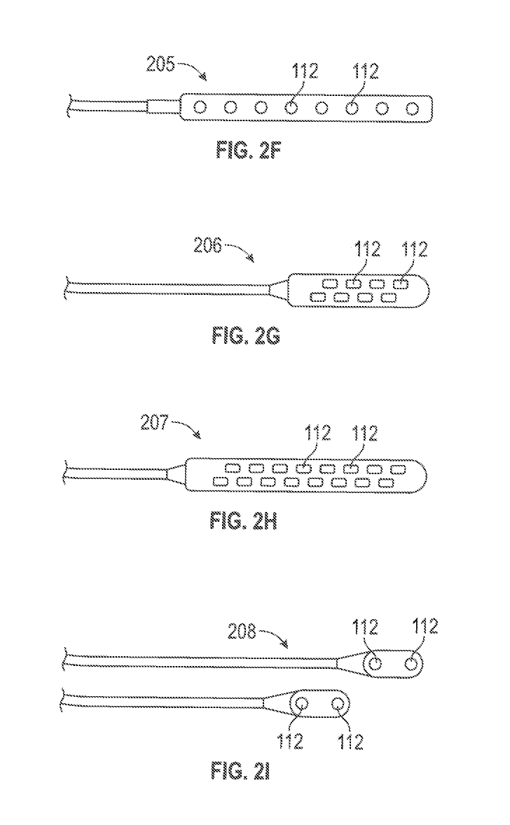

FIGS. 2A-2I, respectively, depict stimulation portions 200-208 for inclusion at the distal end of lead. For example, the stimulation portions 200-208 depict a conventional stimulation portion of a “percutaneous” lead with multiple electrodes 112. The stimulation portions 200-208 depict a stimulation portion including several segmented electrodes 112. Example fabrication processes are disclosed in U.S. patent application Ser. No. 12/895,096, entitled, “METHOD OF FABRICATING STIMULATION LEAD FOR APPLYING ELECTRICAL STIMULATION TO TISSUE OF A PATIENT.” which is incorporated herein by reference. Stimulation portions 204-208 include multiple electrodes 112 on alternative paddle structures than shown in FIG. 1.

In connection to FIG. 1, the lead 110 may comprise a lead body 172 of insulative material about a plurality of conductors within the material that extend from a proximal end of lead 110, proximate to the PG 150, to its distal end. The conductors electrically couple a plurality of the electrodes 112 to a plurality of terminals (not shown) of the lead 110. The terminals are adapted to receive electrical pulses and the electrodes 112 are adapted to apply the pulses to the stimulation target of the patient. It should be noted that although the lead 110 is depicted with twenty electrodes 112, the lead 110 may include any suitable number of electrodes 112 (e.g., less than twenty, more than twenty) as well as terminals, and internal conductors.

Although not required for all embodiments, the lead body 172 of the lead 110 may be fabricated to flex and elongate upon implantation or advancing within the tissue (e.g., nervous tissue) of the patient towards the stimulation target and movements of the patient during or after implantation. By fabricating the lead body 172, according to some embodiments, the lead body 172 or a portion thereof is capable of elastic elongation under relatively low stretching forces. Also, after removal of the stretching force, the lead body 172 may be capable of resuming its original length and profile. For example, the lead body may stretch 10%, 20%, 25%, 35%, or even up or above to 50% at forces of about 0.5, 1.0, and/or 2.0 pounds of stretching force. Fabrication techniques and material characteristics for “body compliant” leads are disclosed in greater detail in U.S. Provisional Patent Application No. 60/788,518, entitled “Lead Body Manufacturing,” which is expressly incorporated herein by reference.

For implementation of the components within the PG 150, a processor and associated charge control circuitry for an IPG is described in U.S. Pat. No. 7,571,007, entitled “SYSTEMS AND METHODS FOR USE IN PULSE GENERATION,” which is expressly incorporated herein by reference. Circuitry for recharging a rechargeable battery (e.g., battery charging circuitry 156) of an IPG using inductive coupling and external charging circuits are described in U.S. Pat. No. 7,212,110, entitled “IMPLANTABLE DEVICE AND SYSTEM FOR WIRELESS COMMUNICATION,” which is expressly incorporated herein by reference.

An example and discussion of “constant current” pulse generating circuitry (e.g., pulse generating circuity 152) is provided in U.S. Patent Publication No. 2006/0170486 entitled “PULSE GENERATOR HAVING AN EFFICIENT FRACTIONAL VOLTAGE CONVERTER AND METHOD OF USE,” which is expressly incorporated herein by reference. One or multiple sets of such circuity may be provided within the PG 150. Different pulses on different electrodes 112 may be generated using a single set of the pulse generating circuitry 152 using consecutively generated pulses according to a “multi-stimset program” as is known in the art. Complex pulse parameters may be employed such as those described in U.S. Pat. No. 7,228,179, entitled “Method and apparatus for providing complex tissue stimulation patterns,” and International Patent Publication Number WO 2001/093953 A1, entitled “NEUROMODULATION THERAPY SYSTEM,” which are expressly incorporated herein by reference. Alternatively, multiple sets of such circuitry may be employed to provide pulse patterns (e.g., tonic stimulation waveform, burst stimulation waveform) that include generated and delivered stimulation pulses through various electrodes of one or more leads 110 as is &so known in the art. Various sets of parameters may define the pulse characteristics and pulse timing for the pulses applied to the various electrodes 112 as is known in the art. Although constant excitation pulse generating circuitry is contemplated for some embodiments, any other suitable type of pulse generating circuitry may be employed such as constant voltage pulse generating circuitry.

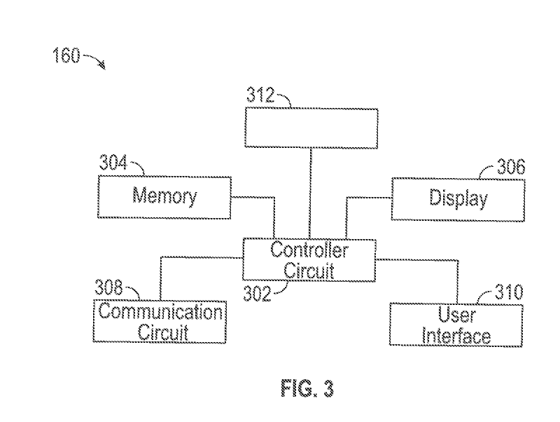

A clinician programmer 160 may be implemented PG 150 to access the memory 161 and to program the PG 150 on the pulse specifications (before and after PG 150 is implanted within the patient). FIG. 3 depicts a schematic block diagram of an embodiment of the monitoring system 160. The monitoring system 160 includes a controller circuit 302 operably coupled to a communication circuit 308, a display 306, a user interface 310, and a memory 304. It may be noted, in alternative embodiments separate devices (not shown) may be employed for charging and/or patient control of the NS system 100.

The controller circuit 302 is configured to control the operation of the clinician programmer 160. The controller circuit 302 may include one or more processors. Optionally, the controller circuit 302 may include a central processing unit (CPU), one or more microprocessors, a graphics processing unit (GPU), or any other electronic component capable of processing inputted data according to specific logical instructions. Optionally, the controller circuit 302 may include and/or represent one or more hardware circuits or circuitry that include, are connected with, or that both include and are connected with one or more processors, controllers, and/or other hardware logic-based devices. Additionally or alternatively, the controller circuit 302 may execute instructions stored on a tangible and non-transitory computer readable medium (e.g., the memory 304).

The communication circuit 308 is configured to receive and/or transmit information with the NS system 100, such as the PG 150. The communication circuit 308 may represent hardware that is used to transmit and/or receive data along a bi-directional communication link. The communication circuit 308 may include a transceiver, receiver, transceiver and/or the like and associated circuitry (e.g., antennas) for wirelessly communicating (e.g., transmitting and/or receiving) with the NS system 100. For example, protocol firmware for transmitting and/or receiving data along the bi-directional communication link may be stored in the memory 304, which is accessed by the controller circuit 308. The protocol firmware provides the network protocol syntax for the controller circuit 308 to assemble data packets, establish and/or partition data received along the bi-directional communication links, and/or the like. The bi-directional communication link may be a wireless communication (e.g., utilizing radio frequency (RF)) link for exchanging data (e.g., data packets) between the one or more alternative medical imaging systems, the remote server, and/or the like. The bi-directional communication link may be based on a standard communication protocol, such as a customized communication protocol, Bluetooth, and/or the like.

Additionally or alternatively, the communication circuit 308 may be operably coupled to a “wand” 165 (FIG. 1). The wand 165 may be electrically connected to a telemetry component 166 (e.g., inductor coil, RF transceiver) at the distal end of wand 165 through respective wires (not shown) allowing bi-directional communication with the PG 150. For example, the user may initiate communication with the PG 150 by placing the wand 165 proximate to the NS system 100. Preferably, the placement of the wand 165 allows the telemetry system of the wand 165 to be aligned with the communication circuit 155 of the PG 150.

The controller circuit 302 is operably coupled to the display 306 and the user interface 310. The display 306 may include one or more liquid crystal displays (e.g., light emitting diode (LED) backlight), organic light emitting diode (OLED) displays, plasma displays, CRT displays, and/or the like. The display 306 may display components of a graphical user interface generated by the controller circuit 302.

The user interface 310 controls operations of the controller circuit 302 and the clinican programmer 160. The user interface 310 is configured to receive inputs from the clinician and/or operator of the clinican programmer 160. The user interface 310 may include a keyboard, a mouse, a touchpad, one or more physical buttons, and/or the like. Optionally, the display 306 may be a touch screen display, which includes at least a portion of the user interface 310 (e.g., via a graphical user interface (GUI)).

Additionally or alternatively, the user interface 310 is configured to allow the user to operate the PG 150. The clinican programmer 160 may be controlled by the user (e.g., doctor, clinician) through the user interface 310 allowing the user to interact with the PG 150. The user interface 310 may permit the user to move electrical stimulation along and/or across one or more of the lead(s) 110 using different electrode 112 combinations, for example, as described in U.S. Patent Application Publication No. 2009/0326608, entitled “METHOD OF ELECTRICALLY STIMULATING TISSUE OF A PATIENT BY SHIFTING A LOCUS OF STIMULATION AND SYSTEM EMPLOYING THE SAME,” which is expressly incorporated herein by reference. Optionally, the user interface 310 may permit the user to designate which electrodes 112 are to stimulate (e.g., emit excitation pulses, in an anode state, in a cathode state) the stimulation target.

Also, the clinican programmer 160 may permit operation of the PG 150 according to one or more spinal cord stimulation (SCS) programs or therapies to treat the patient. For example, the SCS program corresponds to the SCS delivered and/or executed by the PG 150. Each SCS program may include one or mare sets of stimulation parameters of the pulses including pulse amplitude, stimulation level, pulse width, pulse frequency or inter-pulse period, pulse repetition parameter (e.g., number of times for a given pulse to be repeated for respective stimset during execution of program), biphasic pulses, monophasic pulses, etc. The PG 150 may modify its internal parameters in response to the control signals from the clinican programmer 160 to vary the stimulation characteristics of the stimulation pulses transmitted through the lead 110 to the tissue of the patient. NS systems, stimsets, and multi-stimset programs are discussed in PCT Publication No. WO 01/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are expressly incorporated herein by reference.

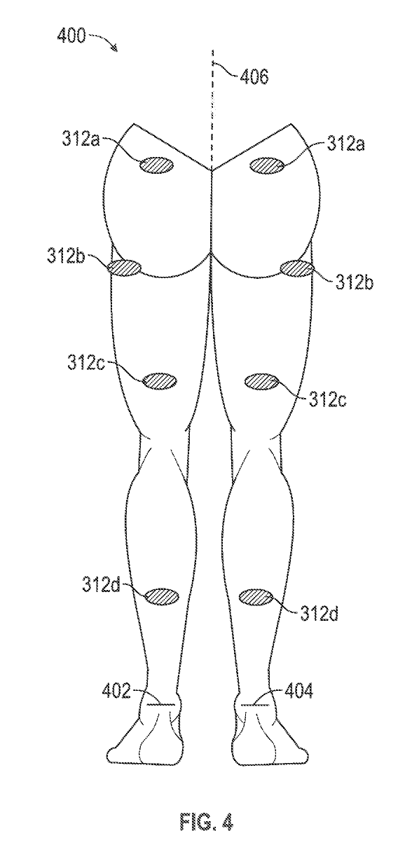

The controller circuit 302 is operably coupled to one or more sets of sensors 312 (e.g., 312a-d). In some embodiments, sensors 312 are coupled to clinician programmer 160 by wire leads connecting to one or more suitable interfaces. In other embodiments, sensors 312 conduct wireless communication with wireless communication circuitry of clinician programmer 160 to permit controller circuit 302 to process the data from the patient's sensed physiological signals. FIG. 4 illustrates a graphical representation of an embodiment of the sets of anatomically opposed sensors 312 positioned bilaterally on a patient 400 at positions that correspond to mirrored anatomical positions of the patient 400 separated by a sagittal plane 406. The sets of sensors 312a-d are positioned at common opposing anatomical positions (e.g., opposing bilateral muscle groups, appendages, limbs, of the patient 400) separated by the sagittal plane 406. For example, the sets of sensors 312a-d are positioned at opposing bilateral muscle groups of the patient 400. The set of sensors 312a are positioned at the gluteus maximus, the set of sensors 312b are positioned at the tensor fasciae latae, the set of sensors 312c are positioned at the quadriceps, and the set of sensors 312d are positioned at the soleus muscle. It may be noted that although four sets of sensors 312 are shown in FIG. 4, in various embodiments more than four and/or less than four sets of sensors 312 (e.g., one pair) may be used. Also, more than one sensor 312a-d may be used at any single position. For example, a set of sensors 312 may include two sensors on a left calf of the patient 400 and two sensors on the right calf of the patient 400.

The sets of sensors 312 are configured to acquire and/or monitor one or more physiological signals of the patient 400. For example, the sets of sensors 312 are configured to measure electromyography (EMG) activity or measurement, skin conductance, blood oxygen saturation, blood pressure, temperature, peripheral nerve activity, and/or the like. The sets of sensors 312 may generate measurement signals, which are received by the controller circuit 302 and stored in the memory 304. For example, the measurement signals may represent an analog and/or digital signal generated by the sets of sensors 312 representative (e.g., based on frequency, amplitude, phase, binary sequence, and/or the like) of the one or more physiological signals acquired and/or monitored by a corresponding sensor 312.

Optionally, the controller circuit 302 may be configured to adjust the measurement signal to determine the one or more physiological signals. For example, the controller circuit 302 may be configured to fitter (e.g., band pass filter, high pass filter, and/or the like), digitize the measurement signal, amplify the measurement signal, and/or the like.

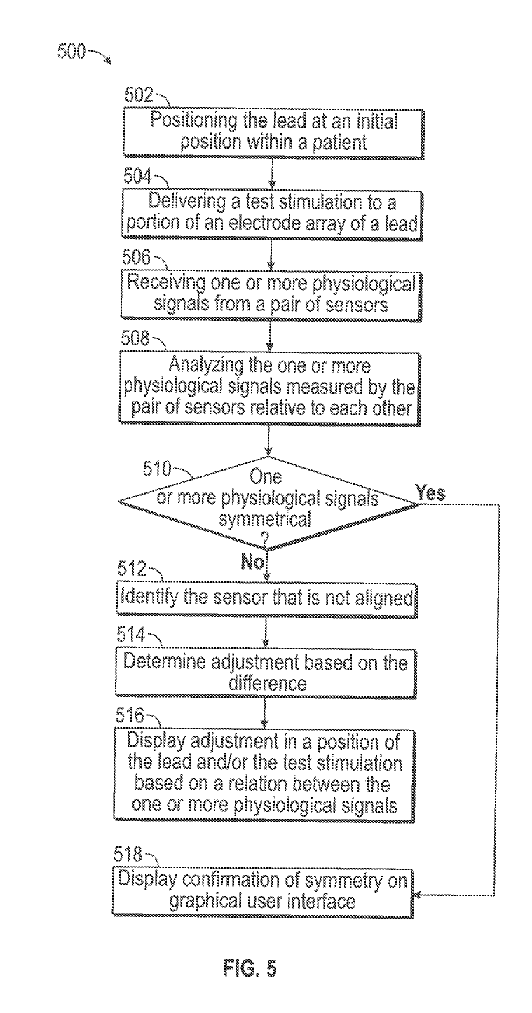

In connection with FIG. 5, the controller circuit 302 is configured to analyze the one or more physiological signals to determine an adjustment in a position of the lead and/or the SCS therapy based on a relation between the one or more physiological signals.

FIG. 5 illustrates a flowchart of an embodiment of a method 500 for electrode monitoring. The method 500, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be reperformed in an iterative fashion. In various embodiments, portions, aspects, and/or variations of the method 500 may be used as one or more algorithms to direct hardware to perform one or more operations described herein.

Beginning at 502 the lead 110 is positioned within the patient 400. For example, the clinician may implant the lead 110 within an epidural space of a dorsal column of a spine of the patient 400.

At 504, the controller circuit 302 instructs the NS system 100 to deliver the SCS therapy to a portion of the electrode array 111 of the lead 110. For example, the controller circuit 302 may receive selections by the clinician of one or more pairs of electrodes 112 of the electrode array 111 from the user interface 310. The clinician may select the electrodes 112 of the one or more pairs of the electrodes 112 as a cathode and/or anode to deliver the SCS therapy. The controller circuit 302 may transmit the selections to the NS system 100 along the bi-directional communication link via the communication circuit 308. Based on the instructions received along the bi-directional communication link, the controller circuit 151 may instruct the switching circuit 157 to selectively connect the selected electrodes 112 to the pulse generating circuitry 152 to deliver the SCS therapy.

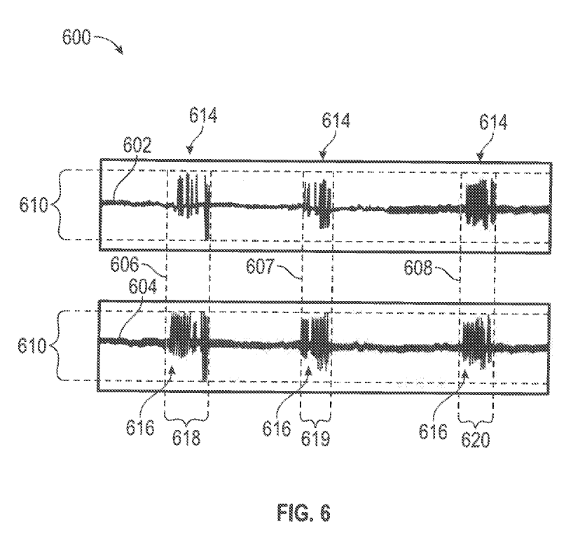

At 506, the controller circuit 302 receives one or more physiological signals from the set of sensors 312. FIG. 6 illustrates a graphical representation 600 of symmetric physiological signals 602, 604. For example, the physiological signals 602, 604 may be sensed by the sets of sensors 312 (e.g., 312a-d). The physiological signals 602, 604 are sensed on opposing sides of a body (e.g., opposing bilateral muscle groups) of the patient 400 about the sagittal plane 406. The physiological signals 602, 604 may correspond to various types of activity such as EMG activity. The physiological signals 602, 604 include a series of pulse trains 614, 616 representing evoked responses in response to the SCS therapy. Optionally, the controller circuit 302 may be configured to process the physiological signals 602, 604. For example, the controller circuit 302 may adjust the physiological signals 602, 604 by filtering (e.g., high pass filter, band pass filter), rectifying, integrating, derivation, and/or the like.

Additionally or alternatively, the controller circuit 302 may be configured to normalize the physiological signals 602, 604. By normalizing the physiological signals 602, 604, intrinsic differences in the strength of the signals are reduced. Intrinsic difference may arise based on differences in position of the set of sensors 312, electrical characteristics of the set of sensors 312 (e.g., impedances, body surface conductivity, patient specific conditions). The controller circuit 302 may normalize the physiological signals 602, 604 to Improve the signal to noise ratio. For example, the controller circuit 302 may be configured to instruct the NS system 100 to repeat the SCS therapy selected at 504 for a predetermined amount and/or number of repetitions. The controller circuit 302 may be configured to perform the normalization based on an amount of noise in the physiological signals, characterization of the stimulation induced artifacts present in the signals (e.g., which may be generated by test stimulations and/or additional stimulations designed for normalization), characterization of electrode tissue impedance, and/or the like. The controller circuit 302 may rescale the physiological signals 602, 604. Additionally or alternatively, the controller circuit 302 may be configured to average the physiological signals 602, 604 over time. The normalization by the controller circuit 302 may be manual based on user selections received from the user interface 310, semi-automatic, and/or automatic.

At 508, the controller circuit 302 analyzes the physiological signals 602, 604 measured by the set of sensors 312 relative to each other. The controller circuit 302 may analyze the pulse trains 614, 616 to characterize the shape. The shape characteristic of interest of the pulse trains 614, 616 may represent a width, energy and/or work, a positon in time (e.g., delay), a peak-to-peak size, principle component analysis, position and/or negative deflection size, and/or the like. The shape characteristics of interest in the pulse trains 614, 616 from opposing bilateral positioned set of sensors 312 are compared with one another (e.g., acquired from the same opposing bilateral muscle groups).

In connection with FIG. 6, the controller circuit 302 compares the physiological signals 602, 604. The controller circuit 302 may compare the physiological signals 602, 604 to determine differences in amplitudes (e.g., peak magnitude, peak-to-peak, and/or the like) and/or phases (e.g., change in time, displacement over time) of the pulse trains 614, 616 relative to each other. Controller circuit 302 may determine whether the measured physiological signals (EMG) from immediately opposing sets of sensors 312 exhibit approximately the same amplitude. If opposing sensors 312 are placed on the patient symmetrically and the test stimulation is applied approximately at the physiological midline, the measured physiological signals from sensors 312 will be approximately equal in amplitude. Also, the timing of firing of the physiological signals in response to test stimulation should be approximately the same. That is, with appropriately placed sensors and test stimulation from an appropriately placed stimulation lead, controller circuit 302 should detect that the onset times of the firing of respective sets of physiological signals from opposing sensors 312 in response to the test stimulation are approximately equal. Deviations from symmetry in the physiological signals from appropriately placed sensors 312 may indicate that the stimulation lead is not located in an intended position relative to the physiological midline. Examples of comparison methods for analyzing physiological signal symmetry between opposing sets of sensors 312 include thresholding, template matching, frequency analysis, patter recognition, machine learning, computer vision, fuzzy logic, and/or the like. Optionally, the controller circuit 302 may use more than one method as described herein to compare the physiological signals 602, 604.

For example, the controller circuit 302 may compare the pulse trains 614, 616 of the physiological signals 602, 604 to identify a difference there between. The difference is compared to a predetermined non-zero threshold stored in the memory 304. The difference may represent a difference in amplitude and/or phase of the physiological signals 602, 604. The predetermined non-zero threshold may be defined by the clinician based on user selections received from the user interface 310. Additionally or alternatively, the predetermined non-zero threshold may be calculated by the controller circuit 302 based on characteristics of the physiological signals 602, 604. For example, the controller circuit 302 may calculate the predetermined non-zero threshold based on a level of noise, digital artifacts, and/or the like corresponding to signal errors of the monitoring system 300.

In another example, the controller circuit 302 may compare the pulse trains 614, 616 to each other based on template matching. For example, the controller circuit 302 may select a first physiological signal 602 as a template. The template may define the signal characteristics of interest, which are compared to a second physiological signal 604. The controller circuit 302 compares the second physiological signal with the first physiological signal (e.g., the template) to determine differences in the signal characteristics of interest.

In some embodiments, controller circuit 302 processes the physiological data from sensors 312 to determine whether the lead is canted or tilted. For example, test stimulation may be applied using respective electrodes in a given column of a paddle lead. Controller circuit 302 processes the physiological data to determine the amplitudes at which physiological responses are first generated for the respective electrodes of the column of the paddle lead. If the amplitudes different by more than a predetermined non-zero threshold, the lead may be tilted or canted (controller circuit 302 may indicate to the clinician to reposition the paddle lead as discussed herein).

In another example, the controller circuit 302 may compare the pulse trains 614, 616 of the physiological signals 602, 604 to each other based on a frequency analysis. For example, the controller circuit 302 may be configured to execute a Fourier transform on the physiological signals 602, 604 to form a frequency domain. The controller circuit 302 may compare frequency peaks of the physiological signals 602, 604. The frequency peaks may indicate frequencies of the pulse trains 614, 616 of the physiological signals 602, 604. The controller circuit 302 may compare positions of the frequency peaks relative to each other. When the frequency peaks of the controller circuit 302 are aligned with each other within the predetermined non-zero threshold (e.g., as described above), the controller circuit 302 may be configured to determine that the physiological signals 602, 604 are in phase with each other. Additionally or alternatively, when the frequency peaks of the controller circuit 302 are not aligned with each other within the predetermined nonzero threshold (e.g., as described above), the controller circuit 302 may be configured to determine that the physiological signals 602, 604 are not phase with each other.

In another example, the controller circuit 302 may compare the pulse trains 614, 616 of the physiological signals 602, 604 to each other based on pattern recognition, computer vision, fuzzy logic, and/or machine learning. The pattern recognition, computer vision, fuzzy logic, and/or machine learning may be based on an artificial neural network, classification, predictive model, and/or the like stored in the memory 304. For example, the pattern recognition, computer vision, fuzzy logic, and/or machine learning may be configured to identify differences in position (e.g., relating to phase) and/or a size (e.g., relating to amplitude) of the pulse trains 614, 616 with respect to each other.

At 510, the controller circuit 302 determines whether the one or more physiological signals 602, 604 are symmetrical with respect to one another. The controller circuit 302 may determine that the one or more physiological signals 602, 604 are symmetrical by comparing the shapes of the pulse trains 614, 616. For example, the shapes of the pulse trains 614, 616 are considered similar or the same when one or more shape characteristics of interest are with the corresponding the predetermined non-zero threshold. The controller circuit 302 may determine that the shapes are similar by comparing at least one of an amplitude, a phase, a length and/or duration, a width, energy and/or work, principle component analysis, position and/or negative deflection size, and/or the like of the pulse trains 614, 616.

For example, the controller circuit 302 may be configured to compare amplitudes 610, 612, phase, lengths 618-620, and/or the like of the pulse trains 614, 616 with each other. The controller circuit 302 may determine whether a difference between the amplitudes 610, 612 of the pulse trains 614, 616 fall within the predetermined non-zero threshold.

In connection with phase, the controller circuit 302 may determine a difference in start time for the pulse trains 614, 616. For example, the controller circuit 302 may identify start firms by identifying a trigger event. The trigger event may correspond to a change in amplitude of the physiological signals 602, 604 representing a start and/or beginning of the pulse trains 614, 616. The controller circuit 302 compares the start times of the pulse trains 614, 616 within windows at 606-608. When the start times for the pulse trains 614 are within a phase threshold of the start times for the pulse trains 616, the physiological signals 602, 604 are in phase with one another. When a difference between the start times for the pulse trains 614 and the start times for the pulse trains 616 exceeds the phase threshold, the physiological signals 602, 604 are out of phase with one another.

In connection with the lengths, the controller circuit 302 may determine a difference in the lengths 618-620 of the pulse trains 614, 616. For example, the controller circuit 302 may identify a start point of the pulse trains 614, 616 based on changes in the amplitude of the physiological signals 602, 604, such as corresponding to the trigger event. The length 618-620 may extend from the start point to an end point. The end point corresponds to when the pulse trains 614, 616 end. For example, the end point may represent when the amplitude of the pulse trains 614, 616 return to the rolling average of the physiological signals 602, 604. The controller circuit 302 may determine the lengths 618-620 of the pulse trains 614, 616 based on a difference between the start point and the end point. When a length of the pulse trains 614 are within a length threshold for a length of the pulse trains 616, the physiological signals 602, 604 have similar lengths with one another. When a difference between the lengths of the pulse trains 614, 616 exceeds the length threshold, the physiological signals 602. 604 do not have similar lengths.

When differences between the amplitudes 610, 612, phase, and lengths 618-620 of the pulse trains 614, 616 fall within corresponding thresholds, flow advances to 518. Otherwise, the flow moves to 512.

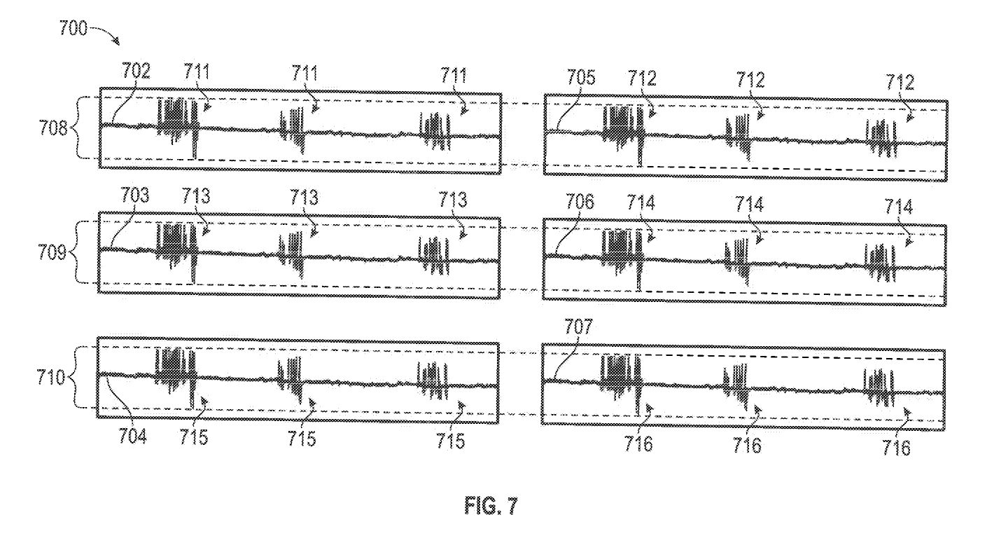

The physiological signals 602, 604 described above in connection with FIG. 6 represent an example of symmetric signals and the related operations by the controller circuit 302. Next, an example embodiment is described in connection with FIG. 7, in which physiologic signals are sensed that are not symmetric. FIG. 7 illustrates graphical representations 700 of an embodiment of a portion of non-symmetric physiological signals 702-707. The physiological signals 702-707 may be sensed by opposing sets of sensors 312. For example, the physiological signals 702, 705 may correspond to the set of sensors 312b, the physiological signals 703, 706 may correspond to the sets of sensors 312c, and the physiological signals 704, 707 may correspond to the sets of sensors 312d. The controller circuit 302 determines differences in the shape characteristics of interest between the physiological signals 702-707 with each other to determine whether the physiological signals 702-707 are symmetric.

For example, the controller circuit 302 may compare the amplitudes 708-710 of the physiological signals 702-707. For example, the controller circuit 302 may calculate a difference between the amplitudes 708 and 709 are within the predetermined non-zero threshold. Since the difference in the amplitudes 708, 709 are within the predetermined non-zero threshold, the controller circuit 302 determines that the amplitudes 708, 709 are similar to and/or the same with respect to each other and are symmetric. The controller circuit 302 may calculate a difference between the amplitude 710 and the amplitudes 708 or 709. The difference between the amplitude 710 and the amplitudes 708 or 709 exceeds the predetermined non-zero threshold. Since the difference in the amplitude 710 and the amplitudes 708 or 709 exceeds the predetermined non-zero threshold, the controller circuit 302 determines that the signals 702-707 are not symmetric.

Since the controller circuit 302 determines that the one or more physiological signals 702-707 are not symmetric, then at 512 (FIG. 5), the controller circuit 302 identifies the set of sensors 312 that is not aligned. Alignment may represent when at least one of the physiological signals 702-707 are not symmetric with each other. The controller circuit 302 identified the amplitude 710 of the signals 704 and 707, which are not symmetric with the amplitudes 708-709. The controller circuit 302 identifies the set of sensors 312d that sense the signals 704, 707.

At 514, the controller circuit 302 may be configured to determine an adjustment based on the difference between the one or more physiological signals for the non-symmetry. The adjustment may correspond to an adjustment in position of the lead 110 within the patient 400 and/or the electrodes 112 that deliver the SCS therapy to increase a probability of symmetry. The controller circuit 302 determines an adjustment of the lead 110 and/or the SCS therapy to modify the characteristics of the pulse trains 711-716 with respect to each other.

The physiological signals 704 and 707 were identified by the controller circuit 302, at 512, as not being symmetric with the physiological signals 702-703, 705-7060. The controller circuit 302 may determine the adjustment based on a position of the set of sensors 312d corresponding to the physiological signals 704. 707 and the shape characteristics of interest relative to the remaining physiological signals 702-703, 705-706.

For example, the controller circuit 302 determines a position of the lead 110 within the epidural space. When physiological signals 702-707 sensed by sets of opposing sensors 312 are symmetric, the controller circuit 302 may determine that the lead 110 is aligned (e.g., not crooked) in the epidural space. The controller circuit 302 determined that the physiological signals 702-703 and 705-706 that are measured by the sets of signals 312b-c are symmetric. Based on the symmetry of the physiological signals 702-703, 705-706 sensed by the set of opposing sensors 312b-c, the controller circuit 302 determined that the lead 110 is not crooked. The adjustment to the lead 110 and/or the SCS therapy determined by the controller circuit 302 must adjust the amplitude 710 of the physiological signals 704, 707. The physiological signals 704, 707 are sensed by the set of sensors 312d, which are positioned proximate to feet of the patient 400. The controller circuit 302 may determine that the lead 110 and/or the SCS therapy must be adjusted lower relative to the epidural space. For example, the lead 110 and/or the SCS therapy must be adjusted closer to nerves corresponding to the feet to increase the amplitudes 710 of pulse trains 715-716.

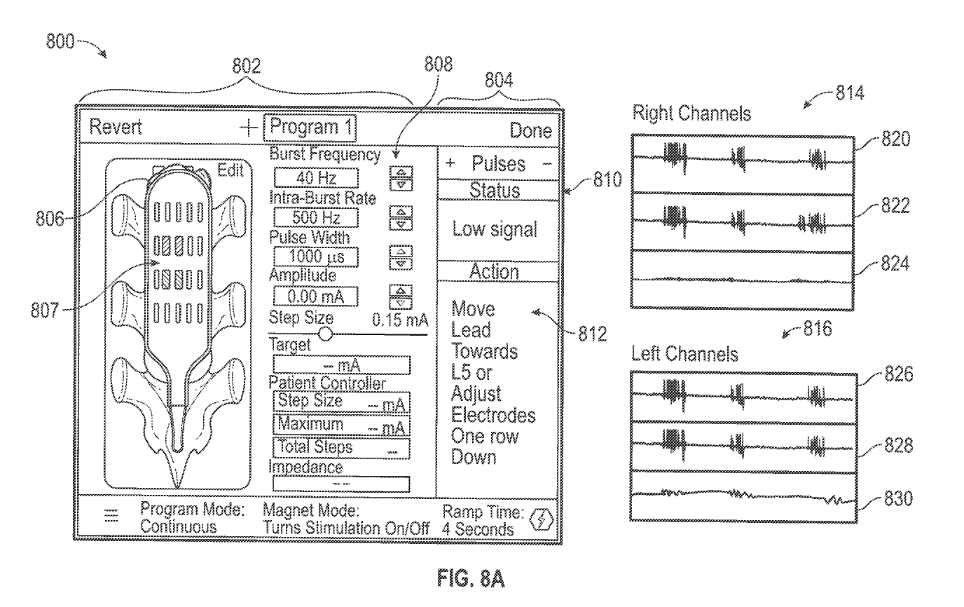

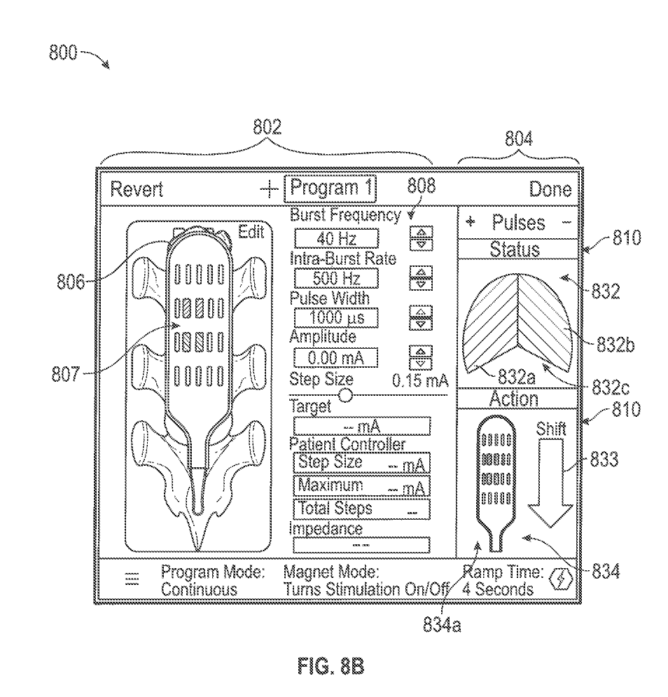

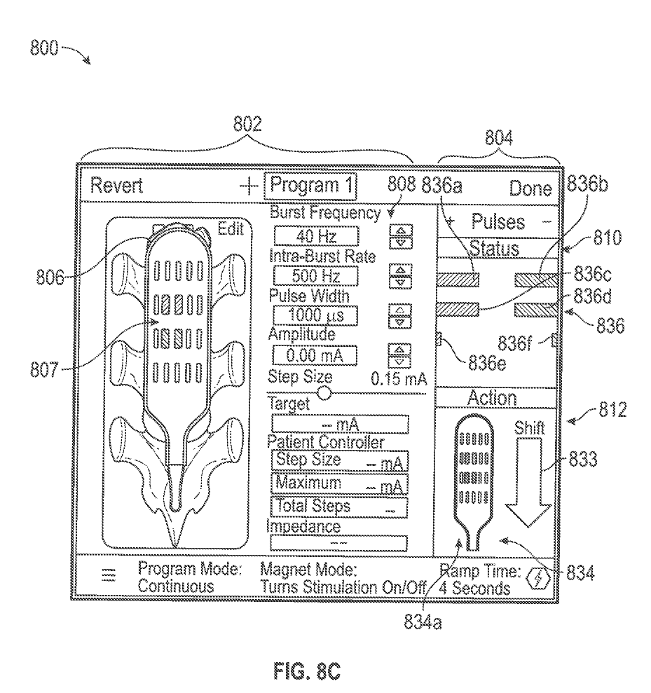

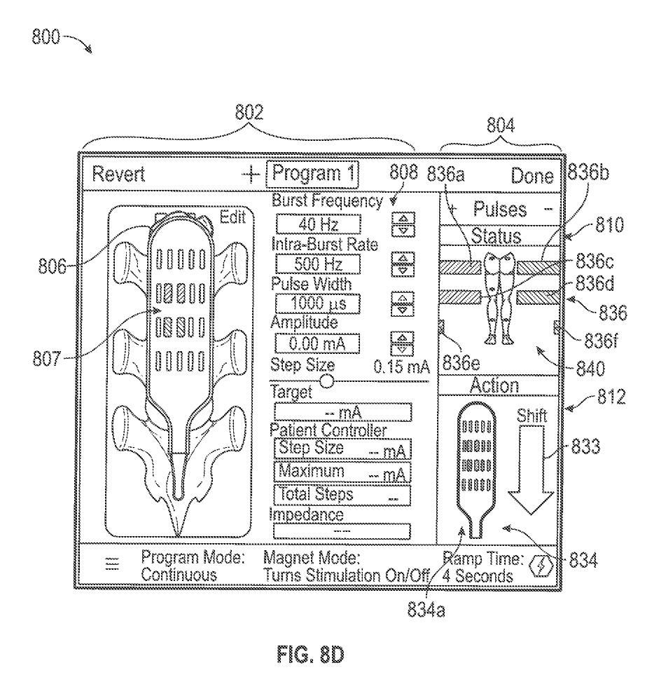

At 516, the controller circuit 302 may be configured to display the adjustment in the position of the lead 110 and/or the SCS therapy based on a relation between the one or more physiological signals 702-707. FIGS. 8A-D illustrates embodiments of user interface components shown on a graphical user interface (GUI) 800 shown on the display 306. The GUI 800 includes a SCS configuration window 802 and an indicator window 804. The SCS configuration window 802 may include a plurality of user interface components 808 to adjust the SCS therapy. For example, the user interface components 808 may enable the clinician to adjust a frequency, amplitude, pulse width, and/or the like of the SCS therapy delivered by the electrode array 111.

Optionally, the SCS configuration window 802 may include a graphical icon 806 representing a position of the lead 110 within the patient 400. The position may be determined based on the physiological signals 702-707. For example, based on the determined symmetry by the controller circuit 302 of the physiological signals 702-703, 705-706 the controller circuit 302 may determine that the lead 110 is aligned within the epidural space of the patient 400. The graphical icon 806 may include an electrode indicator 807. The electrode indicator 807 may be configured to indicate which electrodes 112 within the electrode array 111 are utilized for delivering the SCS therapy.

The indicator window 804 is configured to indicate to the clinician adjustments to the lead 110 and/or the SCS therapy. The indicator window 804 may include a status window 810. The status window 810 may include textual information on a reason for the lack of symmetry between the physiological signals 702-707. For example, such as a low signal and/or low amplitude, a description and/or location of the set of sensors 312d corresponding to the physiological signals 704, 707, and/or the like.

The indicator window 804 may include an action window 812. The action window 812 is configured to indicate to the clinician an action to be taken to adjust the lead 110 and/or the SCS therapy to increase a chance of achieving symmetry between the physiological signals 702-707. For example, the action window 812 may include textual information on a direction to move the lead 110 and/or how to adjust the SCS therapy.

Additionally or alternatively, the GUI 800 may display the physiological signals 814, 816. For example, the physiological signals 814, 816 may he shown as graphical waveforms 820, 822, 824, 826, 828, 830. The graphical waveforms 820, 822, 824, 826, 828, 830 may indicate to the clinician differences between the physiological signals 702-707 acquired by the sets of sensors 312. Optionally, the graphical waveforms 820, 822, 824, 826, 828, 830 may be grouped into bilateral groups to indicate a side of the patient corresponding to a position relative to the patient 400 of the sets of sensors 312.

It may be noted in embodiments, the GUI 800 may include additional graphical icons to indicate to the clinician adjustments and/or status of the SCS therapy.

In connection with FIG. 8B, the status indicator 810 may include a graphical icon 832, such as a pie chart, configured to indicate to the clinician an asymmetry between the physioiogical signals 702-707. For example, the graphical icon 832 may be subdivided representing corresponding sides of the patient 400. The graphical icon 832a may correspond to a right side of the patient 400, and the graphical icon 832b may correspond to a left side of the patient 400. The graphical icons 832a-b are configured to indicate the symmetry of the physiological signals 702-707. For example, the graphical icon 832 may include a void 832c and/or absence of the graphical icons 832a-b indicating a position of the sets of sensors 312 that are not symmetrical.

The action window 812 is shown having graphical indicators 833 and 834 to indicate actions to be taken by the clinician to increase a probability of symmetry of the physiological signals 702-707. The graphical indicator 833 is configured to indicate an adjusted to a position of the lead 110. For example, the graphical indicator 833 is shown as an arrow configured to indicate a direction to reposition the lead 110. Optionally, the graphical indicator 833 may Include textual information, such as a length, degree, and/or the like.

The graphical indicator 834 is configured to indicate an adjustment in the SCS therapy. For example, the graphical indicator 834 may include an electrode indicator 834a configured to highlight an adjustment in the electrodes 112 for delivering the SCS therapy. The electrode indicator 834a is shown shifting the delivery of the SCS therapy by a row corresponding to the direction of the graphical indicator 833.

In connection with FIG. 8C, the status indicator 810 may include a graphical icons 836 (e.g., 836a-f), such as a bar chart configured to indicate to the clinician a symmetry between the physiological signals 702-707. For example, the graphical icons 836 may represent corresponding sides of the patient 400 and/or sets of sensors 312. The graphical icons 836 include bars 836a-f representing the one or more physiological signals acquire by the sets of sensors 312. For example, the bars 836a-b may correspond to the one or more physiological signals acquired by the sensor 312b, the bars 836c-d may correspond to the one or more physiological signals acquired by the sensor 312c, and the bars 836e-f may correspond to the one or more physiological signals acquired by the sensor 312d. A position of the bar (e.g., left, right) may correspond to a bilateral &de of the patient 400 the sets of sensors are located 312. Differences in a size of the bars 836a-f are configured to indicate an asymmetry of the one or more physiological signals. For example, when the bars 836a-f are a similar and/or the same size may indicate that the one or more physiological signals are symmetrical. Alternatively, when a size of the bars 836a-f is different relative to the remaining bars 836a-f this may indicate that the one or more physiological signals are not symmetrical. For example, a size of the bars 836a-d are similar to and/or the same size with respect to each other, which indicates that the one or more physiological signals of the sets of sensors 312b-c are symmetrical. A size of the bars 836e-f are different with respect to the bars 836a-d, which indicates the one or more physiological signals of the set of sensors 312d is not symmetrical with the sets of sensors 312b-c.

In connection with FIG. 8D, the status indicator 810 may include a graphical icon 840 configured to indicate a position of the sets of sensors 312 corresponding to the bars 836a-f. For example, the graphical icon 840 is a representation of the patient 400 having positions of the sets of sensors 312. Each of the bars 836a-f are positioned relative to the graphical icon 840 corresponding to a position of the sets of sensors 312 that are representative of the one or more physiological signals of the bars 836a-f.

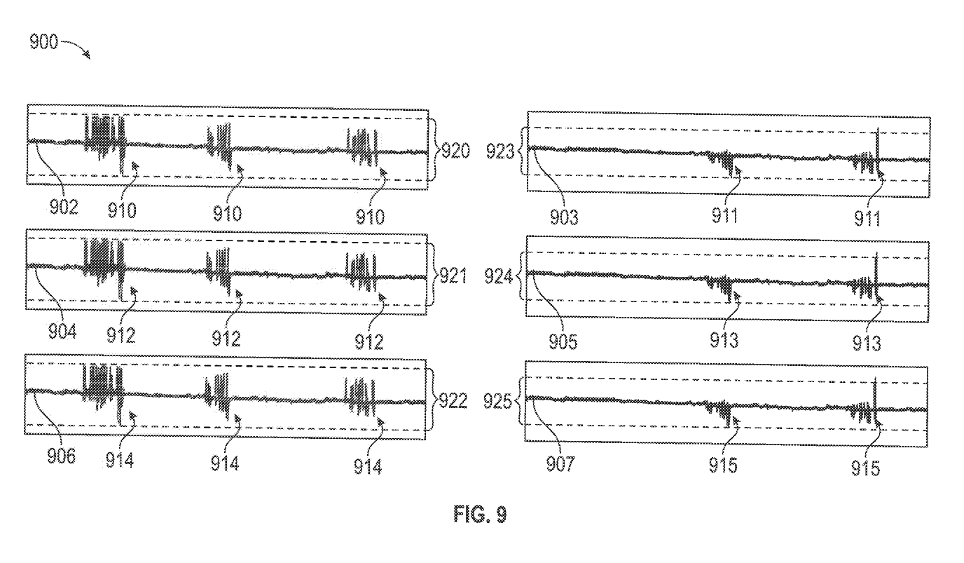

In connection with FIG. 9, the controller circuit 302 may be configured to indicate that the lead 110 needs to be rotated. FIG. 9 illustrates graphical representations 900 of an embodiment of a portion of a plurality of physiological signals 902-907. The physiological signals 902-907 may correspond to opposing sets of sensors 312. For example, the physiological signals 902-907 may correspond to the set of sensors 312b-d. The physiological signals 902, 904 and 906 may correspond to one of the size of the patient 400 and the physiological signals 903, 905 and 907 may indicate that an opposing bilateral side of the patient. The controller circuit 302 may compare the physiological signals 902-907 with each other to determine if the physiological signals 902-907 are symmetric.

For example, the controller circuit 302 may determine that the physiological signals 902-907 are not symmetric based on difference in the amplitudes 920-925 of the pulse trains 910-915. For example, the controller circuit 302 may determine that the amplitudes 920-922 are similar to and/or the same within the predetermined non-zero threshold, but the amplitude 923-925 is smaller than the amplitudes 920-922. Based on the differences in amplitudes 920-925, the controller circuit 302 may determine that the physiological signals 903, 905, and 907 are not symmetrical with the physiological signals 910, 912, and 914.

Based on the physiological signals 903, 905, and 907 corresponding to one of the bilateral sides of the patient 400, the controller circuit 302 may determine that the lead 110 is not aligned within the epidural space. For example, the physiological signals 910, 912, 914 are symmetric (e.g., have similar lengths, amplitude, phase, and/or the like within the predetermined non-zero threshold). Based on the asymmetric determination by the controller circuit 302 for the remaining physiological signals 903, 905, 907, the controller circuit 302 may determine that the lead 110 is not-aligned (e.g., crooked) in the epidural space.

The controller circuit 302 may be configured to determine an adjustment based on the difference in the physiological/signals 902-907. The adjustment may correspond to an adjustment in position of the lead 110 within the patient 400. The adjustment may be based on the characteristics of the pulse trains 910-915 with respect to each other, and a position of the corresponding set of sensors 312 with respect to the patient 400. For example, the controller circuit 302 may be configured to determine an amount to rotate the lead 110 based on a difference in the amplitudes 920-925. The controller circuit 302 may determine a difference in magnitudes of the amplitudes 920-922 relative to the amplitudes 923-925. Based on the difference in magnitudes the controller circuit 302 may determine an amount to rotate the lead 110 within the epidural space of the patient 400.

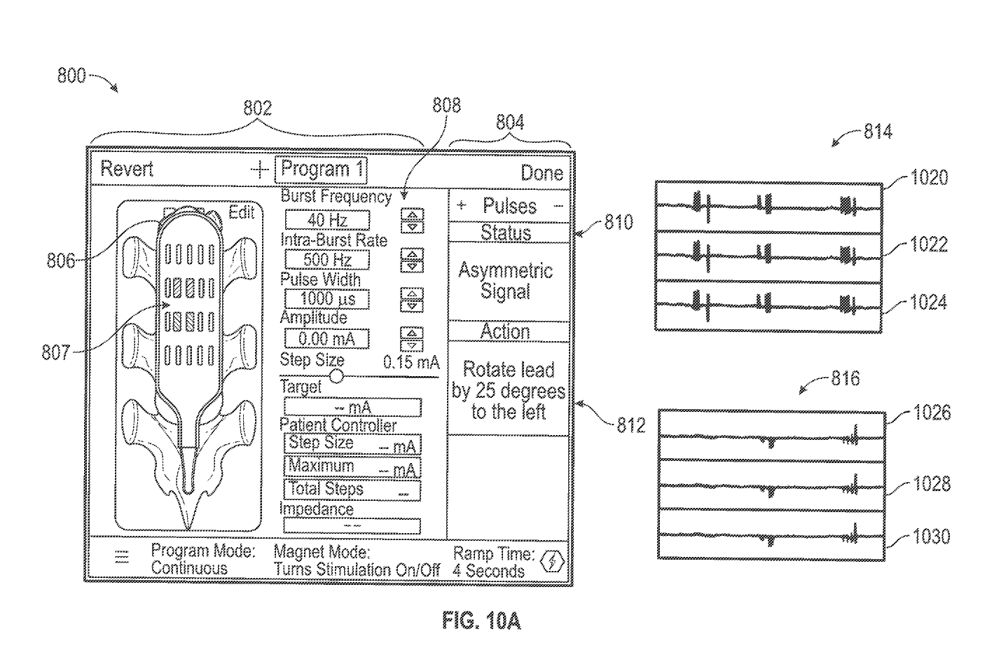

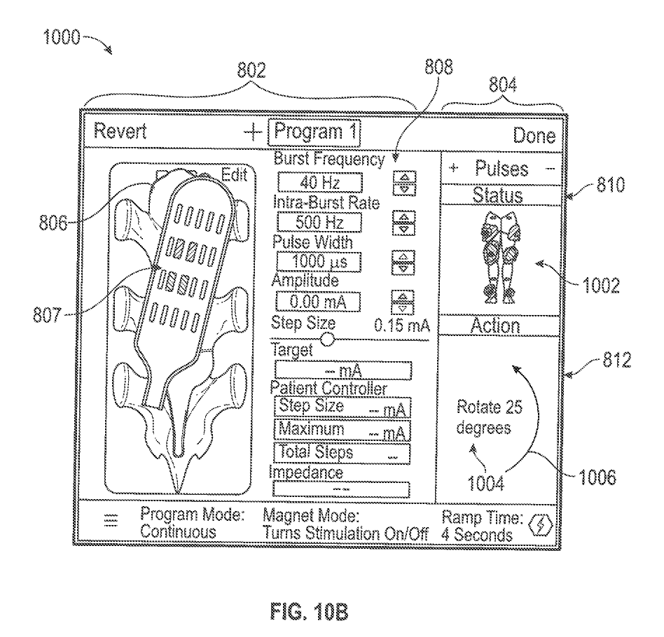

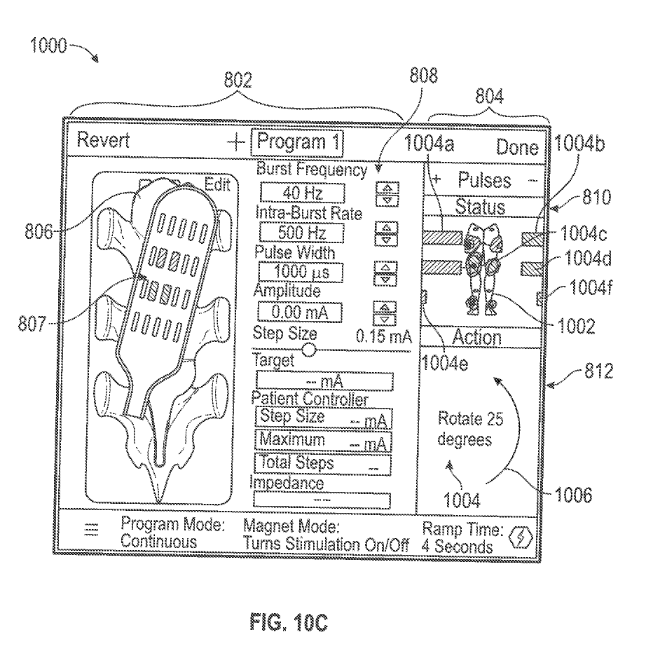

In connection with FIGS. 10A C, the controller circuit 302 may be configured to display the adjustment in the position of the lead 110 based on a relation between the one or more physiological signals 902-907. FIGS. 10A-C illustrates embodiments of user interface components shown on a GUI 1000 shown on the display 306. Similar to and/or the same as the GUI 800 shown in FIGS. 8A-D, the GUI 1000 includes a SCS configuration window 802 and an indicator window 804. The SCS configuration window 802 includes the graphical icon 806 representing a position of the lead 110 within the patient 400. As shown in the GUI 1000, the position may be determined based on the physiological signals 902-907. For example, based on the determined symmetry by the controller circuit 302 of the physiological signals 902, 904, 906 the controller circuit 302 may determine that the lead 110 is not-aligned within the epidural space of the patient 400. The graphical icon 806 is shown being rotated and/or not-aligned within the epidural space.

Similar to and/or the same as the indicator window 804 shown in FIG. 8A, the status window 810 and the action window 812 may include textual information on a reason for the lack of symmetry between the physiological signals 902-907. For example, such as an indication on difference in the physiological signals 902-907, a description and/or location of the sets of sensors 312 not symmetrical, and/or the like. The action window 812 includes textual information on a direction to move the lead 110.

Additionally or alternatively, the GUI 1000 may display the physiological signals 814, 818. For example, the physiological signals 814, 816 may be shown as graphical waveforms 1020, 1022, 1024, 1026, 1028, 1030. The graphical waveforms 1020, 1022, 1024, 1026, 1028, 1030 may indicate to the clinician differences between the physiological signals 902-907 acquired by the sets of sensors 312. Optionally, the graphical waveforms 1020, 1022, 1024, 1026, 1028, 1030 may be grouped into bilateral groups to indicate a side of the patient corresponding to a position relative to the patient 400 of the sets of sensors 312.

Similar to and/or the same as the GUI 800, the GUI 1000 may include additional graphical icons to indicate to the clinician adjustments and/or status of the SCS therapy.

In connection with FIG. 10B, the status indicator 810 may include a graphical icon 1002, such as a heat map, configured to indicate to the clinician an asymmetry between the physiological signals 902-907. For example, the graphical icon 1002 may represent characteristics of the one or more physiological signals 902-907 at a position of the sets of sensors 312 relative to the patient 400. The graphical icon 1002 may include a color coding that includes red, yellow, green, and/or the like, which are configured to indicate a strength of the physiological signals 902-907 measured at the sets of sensors 312. The color coding may be predefined based on set characteristics (e.g., amplitude, phase, frequency, and/or the like) stored in the memory 304. Additionally or alternatively, the color coding may be defined by the clinician based on user selection received from the user interface 310. The color coding of the graphical icon 1002 may indicate to the clinician symmetry and/or asymmetrical evoked responses based on the physiological signals 902-907.

For example, red may indicate a large evoked response represented as the pulse train (e.g., the pulse trains 910, 912, 914) based on an amplitude 920-922 of the physiological signals 902, 904, 906. The graphical icon 1002 indicates that the red is on a single bilateral side of the patient 400. For example, a lack of red may on a single side of the patient 400 indicates misalignment of the lead 110 within the epidural space.

The action window 812 is shown having graphical indicator 1002 and textual information 1004 to indicate actions to be taken by the clinician to increase a probability of symmetry of the physiological signals 902-907. The graphical indicator 1006 is configured to indicate an adjusted to a position of the lead 110. For example, the graphical indicator 1006 is shown as an arrow configured to indicate a direction and/or rotation to reposition the lead 110.

In connection with FIG. 10C, the status indicator 810 may include a graphical icons 1004 (e.g., 1004a-f), such as a bar chart, configured to indicate to the clinician a symmetry between the physiological signals 902-907. For example, the graphical icons 1004 may represent corresponding sides of the patient 400 and/or sets of sensors 312. The graphical icons 1004 include bars 1004a-f representing the one or more physiological signals acquire by the sets of sensors 312. For example, the bars 1004a-b may correspond to the one or more physiological signals acquired by the sensor 312b, the bars 1004c-d may correspond to the one or more physiological signals acquired by the sensor 312c, and the bars 1004e-f may correspond to the one or more physiological signals acquired by the sensor 312d. A position of the bar (e.g., left, right) may correspond to a bilateral side of the patient 400 the sets of sensors are located 312. Differences in a size of the bars 836a-f are configured to indicate an asymmetry of the one or more physiological signals. For example, when the bars 1004a-f are a similar and/or the same size may indicate that the one or more physiological signals are symmetrical. Alternatively, when a size of the bars 1004a-f is different relative to the remaining bars 1004a-f may indicate that the one or more physiological signals are not symmetrical. Optionally, the bars 1004a-f may be shown concurrently with the graphical icon 1002. For example, each of the bars 1004a-f are positioned relative to the graphical icon 1002 corresponding to a position of the sets of sensors 312 that are representative of the one or more physiological signals 902-907 of the bars 1004a-f.

Additionally or alternatively, the controller circuit 302 may be configured to indicate that the lead 110 needs to be moved laterally within the epidural space and/or the SCS therapy based on a difference in phases of the pulse trains. For example, a set of physiological signals corresponding to one of the bilateral sides of the patient 400 is shifted in phase with respect to the physiological signals of the opposing bilateral side. The difference in phases identified by the controller circuit 302 correspond to a shift within the epidural space laterally. The adjustment may correspond to an adjustment in position of the lead 110 within the patient and/or the electrodes 112 utilized by the NS system 100 to deliver the SCS therapy to the patient 400. The adjustment may be based on the characteristics of the pulse trains 711-716 with respect to each other, and a position of the corresponding set of sensors 312 with respect to the patient 400. The controller circuit 302 may determine the adjustment of the lead 110 and/or the SCS therapy based on the physiological signals not in phase and/or characteristics of the remaining physiological signals.

The identified set of sensors 312 (e.g., determined at 512), corresponding to the bilateral side of the patient 400 having the physiological signals that are not in phase, the controller circuit 302 may determine to adjust the lead 110 and/or the SCS therapy towards the identified set of sensors 312. The controller circuit 302 may be configured to determine a lateral adjustment based on the phase difference between the physiological signals. The adjustment may be based on the characteristics of the pulse trains with respect to each other, and a position of the corresponding set of sensors 312 with respect to the patient 400. For example, the controller circuit 302 may be configured to determine an amount to adjust laterally the lead 110 based on a difference in the phases. Similar to and/or the same as the adjustment illustrated in the GUI 800 of FIGS. 8A-D and/or the GUI 1000 of FIGS. 10A-C, the controller circuit 302 may indicate via the display 306 that the lead 110 is adjusted in position laterally and/or adjust a column of the electrode array 111 delivered by the SCS therapy.

Returning to FIG. 5, when the one OF more physiological signals 602, 604 are determined to be symmetric, at 518, the controller circuit 302 may be configured to display a confirmation of the symmetry on the GUI. For example, the controller circuit 302 may display in the status window 810 may Include textual information that the one or more physiological signals are symmetric.

It may be noted that the various embodiments may he implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” “controller circuit,” “circuit,” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “controller circuit”.

The computer, subsystem, controller circuit, circuit execute a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, subsystem, controller circuit, and/or circuit to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first” “second,” and “third,” etc. are used merely as labels, and are not intended to impose numerical requirements on their objects. Further, the limitations of the following claims are not written in means—plus-function format and are not intended to be interpreted based on 35 U.S.C., § 112(f), unless and until such claim limitations expressly use the phrase “means for” followed by a statement of function void of further structure.

What is claimed is:

1. A clinician programmer device for controlling a neurostimulation system including a pulse generator and a stimulation lead during an implant procedure, comprising:
a controller for controlling the device according to executable instructions;
a display for providing a user interface to a user of the device;
memory for storing executable instructions and data; and
circuitry for communicating with a plurality of sensors for sensing physiological signals from a patient when the plurality of sensors are positioned bilaterally on the patient;
wherein the controller is configured to execute instructions stored in the memory to: (1) communicate with the pulse generator to apply spinal cord stimulation to the patient through the stimulation lead, (2) communicate with the plurality of sensors to acquire physiological data from the patient occurring in response to the spinal cord stimulation, (3) process the acquired physiological data to identify non-symmetry of the patient's physiological response detected using at least one set of bilaterally positioned sensors of the plurality of sensors, (4) identify a potential spatial adjustment of the stimulation lead to reduce or eliminate the non-symmetry of the patient's physiological response, and (5) display an indication of the spatial adjustment on the display to the user of the device.

2. The device of claim 1, wherein the controller is configured to execute instructions to identify differences in amplitude in physiological data from respective sensors of the at least one set of bilaterally positioned sensors.

3. The device of claim 1, wherein the controller is configured to execute instructions to identify differences in phase in physiological data from respective sensors of the at least one set of bilaterally positioned sensors.

4. The device of claim 1, wherein the controller is configured to identify differences in onset time of the patient's physiological response in physiological data from respective sensors of the at least one set of bilaterally positioned sensors.

5. The system of claim 1 wherein the controller circuit is configured to display a user interface component depicting the patient's physiological response and the user interface component includes an indication of respective sets of physiological data, detected using the at least one set of bilaterally positioned sensors, relative to each other.

6. The system of claim 5, wherein the user interface component is a gauge, a bar graph, a graph, an arrow, textual information, a graphical icon, a heat map, a pie chart.

7. The system of claim 1, wherein the controller circuit is configured to execute a configuration phase of operation to apply spinal cord stimulation for normalization of physiological signals before identification of spatial adjustment of the stimulation lead is performed.

8. The system of claim 1, wherein the plurality of sensors are adapted for positioning at an opposing bilateral muscle group of the patient, wherein the opposing bilateral muscle group includes a gluteus maxims, tensor fasciae latae, quadriceps, or soleus muscle.

9. The system of claim 1, wherein the patient's physiological response is determined by measuring electromyography activity.

10. The system of claim 1 wherein the controller is configured to detect whether the stimulation lead is positioned in a canted orientation within an epidural space of the patient and to identify a spatial adjustment on the display to move the stimulation lead to reduce or eliminate the canted orientation.

11. A clinician programmer device for controlling a neurostimulation system including a pulse generator and a stimulation lead during an implant procedure, comprising:
a controller for controlling the device according to executable instructions;
a display for providing a user interface to a user of the device;
memory for storing executable instructions and data:
wireless communication circuitry connected to the controller, wherein the controller is configured to wirelessly communicate with a plurality of sensors using the wireless communication circuitry to obtain physiological data when the plurality of sensors are positioned bilaterally on the patient; and
wherein the controller is configured to execute instructions stored hi the memory to: (1) communicate with the pulse generator to apply spinal cord stimulation to the patient through the stimulation lead, (2) communicate with the plurality of sensors to acquire physiological data from the patient occurring in response to the spinal cord stimulation, (3) process acquired the physiological data to identify non-symmetry of the patient's physiological response detected using at least one set of bilaterally positioned sensors of the plurality of sensors, (4) identify a potential spatial adjustment of the stimulation lead to reduce or eliminate the non-symmetry of the patient's physiological response, and (5) display an indication of the spatial adjustment on the display to the user of the device.

12. The device of claim 11, wherein the controller is configured to execute instructions to identify differences in amplitude in physiological data from respective sensors of the at least one set of bilaterally positioned sensors.

13. The device of claim 11, wherein the controller is configured to execute instructions to identify differences in phase in physiological data from respective sensors of the at least one set of bilaterally positioned sensors.

14. The device of claim 11, wherein the controller is configured to identify differences in onset time of the patient's physiological response in physiological data from respective sensors of the at least one set of bilaterally positioned sensors.

15. The system of claim 11, wherein the controller circuit is configured to display a user interface component depicting the patient's physiological/response and the user interface component includes an indication of respective sets of physiological data, detected using the at least one set of bilaterally positioned sensors, relative to each other.

16. The system of claim 15, wherein the user interface component is a gauge, a bar graph, a graph, an arrow, textual information, a graphical icon, a heat map, a pie chart.

17. The system of claim 15, wherein the controller circuit is configured to execute a configuration phase of operation to apply spinal cord stimulation for normalization of physiological signals before identification of spatial adjustment of the stimulation lead is performed.

18. The system of claim 11, wherein the plurality of sensors are adapted for positioning at an opposing bilateral muscle group of the patient, wherein the opposing bilateral muscle group includes a gluteus maximus, tensor fasciae latae, quadriceps, or soleus muscle.

19. The system of claim 11, wherein the patient's physiological response is determined by measuring electromyography activity.

20. The system of claim 11, wherein the controller is configured to detect whether the stimulation lead is positioned in a canted orientation within an epidural space of the patient and to identify a spatial adjustment on the display to move the stimulation lead to reduce or eliminate the canted orientation.
